(19) United States
(12) Patent Application Publication
(10) Pub. No.: US 2006/0207351 A1
(43) Pub. Date: Sep. 21, 2006

(54) Equipment and method for collecting trace substances in the air

(76) Inventors: Zuhong Lu, Nanjing (CN); Zuming Tang, Nanjing (CN); Jllun Zhu, Nanjing (CN); Yuzhi Yang, Nanjing (CN)

Correspondence Address: JACOBSON HOLMAN PLLC, 400 SEVENTH STREET N.W., SUITE 600, WASHINGTON, DC, 20004, US

(21) Appl. No.: 10/555,006

(22) PCT Filed: Apr. 26, 2004
(86) PCT No.: PCT/CN04/00398

§ 371 Date: Oct. 31, 2005

(30) Foreign Application Priority Data
May 1, 2003 (CN) 03113389.4

(51) Int. Cl. G01N 1/24; G01N 33/50

(52) U.S. Cl. 73/863.21; 73/864.34; 436/181

(57) ABSTRACT

The device for sampling the air-brone trace-particulates is a device to collect and gather pathogens and trace chemical substances especially micro pathogens such as virus particles in the air. The device is composed of the air-collecting cavity (a) which is filled with viscous molecular materials, the valve (b), the eluent container (d) and the air pump (J1 or J3). The air-collecting cavity has an air intake and an air outlet. The air intake is linked to the eluent container through a peristaltic pump and to the atmosphere (c) through the valve. The outlet is connected to the air pump and to the gathering container (f) through the valve. The collecting method is as follows: after the pump (J1), the two valves (b4) and (b1) are opened, the air sample is then collected and gathered in the air-collecting cavity. The volume of collected air is measured with the flow meter (i). When the volume meets the specified standard, the pump (J1), the two valves (b4) and (b1) are all closed. Then the accessorial washing device named ultrasonator (h) is turned on, the pump (J2), the three valves (b3), (b5) and (b6) are all turned on. The pump (J2) forces the eluent pass the gathering area and elutes the component in the air in the gathering area.

This is a nationalization application under 35 U.S.C. 371 of PCT/CN2004/000398, filed Apr. 26, 2004 and published in Chinese.

### 1. FIELD OF THE INVENTION

The invention is a device that is to collect and gather pathogens and trace chemical substances in the air, especially some tiny pathogens such as virus.

### 2. BACKGROUND OF THE INVENTION

To collect and detect the pathogens and harmful components in the air is of great significance to the control of respiratory epidemics, the exploration on biochemical warfare reagents and the surveillance over pathogens and toxic chemical substances in sensitive areas. Currently, the microporous filter membrane and the negative pressure filtration are used to collect and concentrate the pathogens. However, as the aperture of microporous filter membrane is at the micro-level, it cannot be used to collect virus and chemical molecules of trace amounts; besides, as the small aperture of the microporous filter membrane fails to generate the high flowing speed, the quantity of the air for detecting and analyzing is very small. For example, the Severe Acute Respiratory Syndrome (SARS), once prevalent in Asia, is considered to be an infectious disease of great harm caused by coronal virus that is spread by means of air. The development of a method or a device that may directly monitor the virus in the air and its content is the key to the prevention and control of the disease, and is also an international problem that is urged to be solved at present. Nevertheless, the collectors available nowadays of the air pathogens can only collect virus, and they cannot gather and detect virus whose diameter is of several nanometer. As a result, in order to realize the high-efficient and quick on-the-spot detection of the SARS virus in the air, it is urgent to develop a method or a device that can sample, concentrate and detect the virus in the air and also could be installed in hospitals, malls and bus or railway stations which are sensitive to the virus.

### 3. DESCRIPTION OF THE INVENTION

### (1) Technical Problem

On the basis of the requests mentioned above, the invention aims to propose a collecting method and the corresponding device. It can be used to collect and concentrate the trace chemical substances and the pathogens including virus in the air quickly and efficiently.

### (2) The Technical Scheme

The device of the invention to collect the air-brone trace-particulates is composed of an air-collecting cavity which is filled with viscous molecular materials, several valves and several air pumps. The air-collecting cavity containing an air intake and an air outlet is connected to the air pump with its two ends open to the atmosphere. The inside wall of the air-collecting cavity is smeared or filled with viscous molecular materials, for example, polymer resin particles, polymer gelatinoid, biomacromolecule such as protein glucide amylon or glycerin, agarose, lipin, or compounds composed of two or more of the above substances. The air-collecting cavity with viscous molecular materials is integrated with an ultrasonator.

The collecting method of the invention is as follows:

(1) After the pump (J1), the two valves (b4) and (b1) are turned on, the air sample is collected and then the air-brone trace-particulates are gathered in the air-collecting cavity (a). After the collecting process, The pump (J1), the two valvea (b4) and (b1) are all closed.

(2) After the accessorial washing device named ultrasonator (h) is turned on, the pump (J2), the two valves (b3) and (b5) are also turned on. The pump (J2) forces the eluent in its container (d) to pass the gathering area in the air-collecting cavity (a). The adherent substances in the gathering area was eluted and stored in the gathering container (f).

The disinfectants such as SDS and Trizol are added to the eluent to secure the collecting process.

The detection agents of pathogens, for example, specific fluorescence indicator, specific electrochemical indicator, modified antibody such as antibodies modified with fluorescence, microbeads and nanoparticles etc, are added to the eluent to detect collected pathogens directly. PCR reagents and a controlled-temperature system are available in the gathering container for direct amplification and detection of pathogens genes.

The air-collecting cavity or the gathering container can be integrated with chemical analytical apparatuses such as electrochemical detector, capillary electrophoresis detector, spectral detector, chromatographic detector to detect the adherent substance directly.

Before gathering the air, the collecting solution can be transported to the air-collecting cavity to renature the air-collecting cavity.

PCR (polymerase chain reaction) reagents and a controlled-temperature system can be integrated with the gathering container for a direct amplification and detection of pathogens genes. The eluting process of the air-collecting cavity can be accelerated by a mechanical vibration method.

### (3) Beneficial Effects

The invention proposes a new method and its corresponding device that can gather and concentrate microchemical substances and pathogens (including virus) in the air efficiently and quickly. The device can be integrated with a chemical detector and a detecting device for pathogens to form an integrative device which can gather, concentrate and detect pathogens. The high efficiency air gathering device can gather such pathogens as virus particles, bacteria or chemical substance in the air about 30 L within 10 minutes, and then concentrate the gathering particles into the buffer solution about 1 ml. The simulation result for gathering and detecting the virus particles in the air suggests that the collecting efficiency is higher than 90%. Up to now, there has no similar device yet.

### 4. DESCRIPTION OF THE FIGURE

FIG. 1 is a sketch of the general structure of the invention. The device has an air-collecting cavity (a) with an air intake (ai) and an outlet (ao). There are 8 valves (b1, b2, b3, b4, b5, b6, b7, b8), an atmosphere inlet (c), an eluent container (d), a gathering container (f), the collecting solution container (g), an ultrasonator (h), a flow meter (i), 4 pumps (J1, J2, J3, J4), an accessorial solution container (k) and a waste liquid area (L).

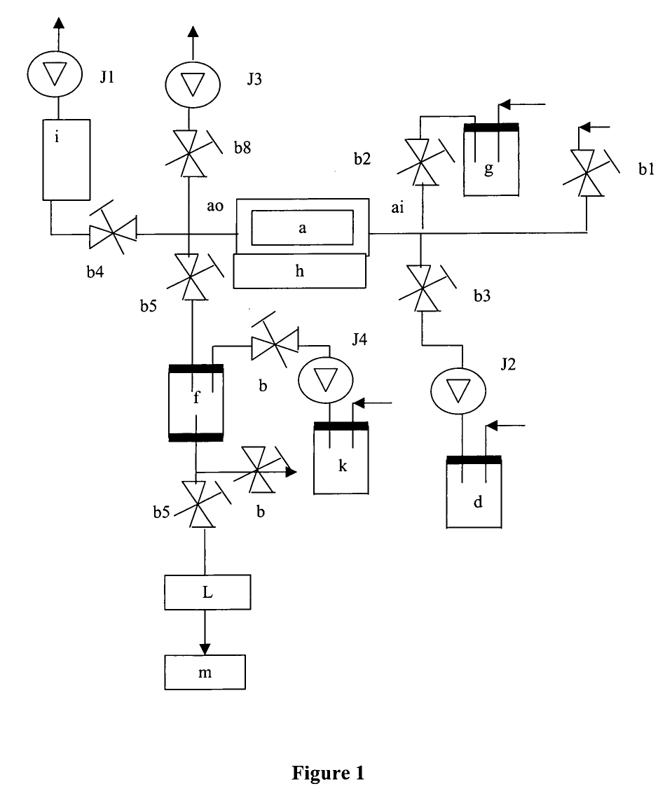

### 5. DETAILED DESCRIPTION

The device realizes its invention purpose through the following technical scheme: The device is composed of an air-collecting cavity (a) which is filled with viscous molecular materials, 5 valves b1, b2, b3, b4 and b5, an eluent container (d), an aspirator pump (J), a gathering container (f), a collecting solution container (g) and a peristaltic pump (J2). The air-collecting cavity has an air intake (ai) and an air outlet (ao). The air intake (ai) is linked to the eluent container (d) through a peristaltic pump (J2) and linked to the atmosphere (c) through the valve (b1). The air outlet (ao) of the air-collecting cavity (a) is connected to the air pump (J1) and to the gathering container (f) through the valve b5.

The air-collecting cavity (a) is a pipe smeared with viscous molecular materials. It can also be filled with such viscous materials as glass fiber, high polymer fiber, porous glass or compounds composed of two or more of the above substances.

Viscous molecular materials in the air-collecting cavity are such viscous substances as polymer resin, polymer gelatinoid, fiberous materials (for instance, glass fiber, nylon and cotton), biomacromolecule (for instance, protein, glucide and amylon), glycerin, agarose and lipin, etc or compounds composed of two or more of the above substances. The air-collecting cavity (a) with viscous molecular materials inside is integrated with an ultrasonator (h).

(1) The air gathering process: Turn on the two valves (b2) and (b8), and then the pump (J3). The collecting solution (g) is transported to the air-collecting cavity (a) to modify or renature the cavity.

(2) After turning off the two valves (b2 and b8) and the pump J3, the pump J1 and the two valves (b4 and b1) are turned on to collect and gather air sample in the air-collecting cavity. The amount of the collected air is measured with a flow meter (i). When the volume meets the specified standard, the pump J1 and the two valves (b4 and b1) are turned off.

(3) After the accessorial washing device named ultrasonator (h) is turned on, the pump J2 and the three valves (b3, b5 and b6) are turned on to force the eluent pass the gathering area and elute the interested components.

(4) After the pump J2 and the two valves (b3 and b5) are turned off, and then the pump J4 and the valve b7 are turned on to transport the accessorial solution to the collecting container (f). The valve b6 is closed and all the solution is transported to the detecting area L. Before a new detection starts, the collecting solution is injected into the air-collecting cavity to renature it.

The detecting reagents of pathogens, for example, specific fluorescence indicator, specific electrochemical indicator, modified antibody such as antibodies modified with fluorescence, microbeads, nanoparticles are added to the eluent to detect collected pathogens directly.

PCR reagents and a controlled-temperature system are available in the gathering container (f) for a direct amplification and detection of pathogens genes.

The air-collecting cavity (a) or the gathering container (f) can be integrated with such chemical analytical apparatuses as electrochemical detector, capillary electrophoresis detector, spectral detector, chromatographic detector to detect adherent substance directly. The elution process is accelerated by the mechanical vibration during eluting the collected substances from the the air-collecting cavity. The technology is of great practical significance for gathering and detecting the air-brone trace-particulates such as microbes, viruses and other chemical compounds.

Referencing to the attached drawings, the using method about the invention is detailed as follows:

(1) The air gathering process: Turn on the two valves (b2) and (b8), and then the pump (J3). The collecting solution (g) is transported to the air-collecting cavity (a) to modify or renature the cavity.

(2) After turning off the two valves (b2 and b8) and the pump J3, the pump J1 and the two valves (b4 and b1) are turned on to collect and gather air sample in the air-collecting cavity. The amount of the collected air is measured with a flow meter (i). When the volume meets the specified standard, the pump J1 and the two valves (b4 and b1) are turned off.

(3) After the accessorial washing device named ultrasonator (h) is turned on, the pump J2 and the three valves (b3, b5 and b6) are turned on to force the eluent pass the gathering area and elute the interested components.

(4) After the pump J2 and the two valves (b3 and b5) are turned off, and then the pump J4 and the valve b7 are turned on to transport the accessorial solution to the collecting container (f). The valve b6 is closed and all the solution is transported to the detecting area L.

(5) The detecting process: Start-up the detecting accessory to detect collected solution and change the solution before a new cycle starts.

(6) The detection results are saved in documental format.

The protein powder labeled with fluorescence is put in the beaker which is placed in an ultrasonator. After the ultrasonator is turned on, the protein powder labeled with fluorescence is vibrated into the air. After that, the device for sampling the air-brone trace-particulates is powered on and the air pump begins to work. The air intake is placed above the ultrasonator about 10 centimeters and the outlet is put in 10 ml water to collect protein molecules that have not been collected by the air-collecting cavity. During the above normal working conditions, the gathering process is completed. Finally, the fluorescence spectrum is used to detect the fluorescence intensity of the solution in the collecting container and the solution in the outlet separately. The ratio between these two intensities can be used to represent the collecting efficiency of the protein particles. In fact, the solution in the collecting container is of high fluorescence intensity, while the solution in the outlet gives no fluorescence signals, which suggests the device can be used to collect protein particles in the air efficiently.

The sterilized HAV powder is put in the beaker which is placed in the ultrasonator. After the ultrasonator is turned on, the virus powder is vibrated into the air. After that, the device for sampling the air-brone trace-particulates is powered on and the air pump begins to work. The air intake is placed above the ultrasonator about 20 centimeters and the outlet is put in 10 ml water to collect virus particles that have not been collected by the air-collecting cavity. During the above normal working conditions, the gathering process is completed. After the collecting process, the samples collected in the air intake and outlet, including the negatrive and positive contrasts, are all added separately to the apertures coated with HAV monoclonal antibody on the plate to react with HAV monoclonal antibody labeled with fluorescence. Finally, the plate is rinsed and the fluorescence intensity is detected. The detection results suggests that there are no fluorescence signals in the apertures of negative contrast and the solution of outlet, while the fluorescence signals are very high in the apertures of positive contrast and the solution of the intake. The ratio of the two fluorescence intensities between the solution in the air-collecting cavity and the solution in the air outlet could be used to evaluate the collecting efficiency of the airborne virus particles of the device. Strong fluorescence are detected in the solution in the collecting container, while no fluorescence signal is detected in the solution of output, which suggests the device could be used to collect virus particles in the air efficiently.

1. A device for sampling the air-brone trace-particulates, which is characterized by an air-collecting cavity (a), a valve and an air pump (J1 or J3). The air-collecting cavity which contains viscous molecular materials inside (a) has an air intake (ai) and an air outlet (ao) and is connected to the air pump (J1 or J3) with its two ends open to the atmosphere (c).

2. According to claim 1, the feature of the device for sampling the air-brone trace-particulates is: the inside wall of the air-collecting cavity (a) is smeared or filled with viscous molecular materials.

3. According to claim 1, the feature of the device for sampling the air-brone trace-particulates is: the viscous molecular materials contained in the air-collecting cavity (a) are composed of such viscous materials as polymer resin particles, polymer gelatinoid, biomacromolecule such as protein, glucide, amylon, glycerin, agarose and lipin etc, or compounds composed of two or more of the above substances.

4. According to claim 1, the feature of the device for sampling the air-brone trace-particulates is: the air-collecting cavity (a) containing viscous molecular materials inside is integrated with an ultrasonator (h).

5. A method to collect the air-brone trace-particulates is characterized by the following procedures:
(i) after the pump (J1), the two valves (b4) and (b1) are turned on, the air sample is collected and then the air-brone trace-particulates are gathered in the air-collecting cavity (a). After the collecting process, The pump (J1), the two valvea (b4) and (b1) are all closed;
(ii) after the accessorial washing device named ultrasonator (h) is turned on, the pump (J2), the two valves (b3) and (b5) are also turned on. The pump (J2) forces the eluent in its container (d) to pass the gathering area in the air-collecting cavity (a); the adherent substances in the gathering area being eluted and stored in the gathering container (f).

6. The method according to claim 5, the feature of the method to collect the air-brone trace-particulates is: Many kinds of reagents for detecting the pathogens can be added to the eluent and make it possible to detect the collected pathogens directly. The reagent may be specific fluorescence indicator, specific electrochemical indicator, modified antibody such as antibodies modified with fluorescence, microbeads, nanoparticles, etc.

7. The method according to claim 5, the feature of the method to collect the air-brone trace-particulates is: PCR reagents and a controlled-temperature system are available in the gathering container (f) for a direct amplification and detection of pathogens genes.

8. The method according to claim 5, the feature of the method to collect the air-brone trace-particulates is: the air-collecting cavity (a) or the gathering container (f) can be integrated with chemical analytical apparatuses such as electrochemical detector, capillary electrophoresis detector, spectral detector, chromatographic detector to detect adherent substance directly.

9. The method according to claim 5, the feature of the method to collect the air-brone trace-particulates is: before gathering the air, the collecting solution in collecting container (g) can be transported to the air-collecting cavity (a) to renature the air-collecting cavity.
